(19) United States
(12) Patent Application Publication
(10) Pub. No.: US 2005/0004017 A1
(43) Pub. Date: Jan. 6, 2005

(54) Methods and compositions for treating hcap associated diseases

(76) Inventors: Yuval Reiss, Kiriat-Ono (IL); Iris Alroy, Ness-Ziona (IL)

Correspondence Address: ROPES & GRAY LLP, ONE INTERNATIONAL PLACE, BOSTON, MA, 02110-2624, US

(21) Appl. No.: 10/490,002

(22) PCT Filed: Sep. 18, 2002
(86) PCT No.: PCT/US02/29577

Related U.S. Application Data
(60) Provisional application No. 60/323,210, filed on Sep. 18, 2001.
(60) Provisional application No. 60/332,350, filed on Nov. 9, 2001.

(51) Int. Cl. A61K 48/00; A61K 38/17

(52) U.S. Cl. 514/12; 514/44

(57) ABSTRACT

The disclosure provides, among other things, methods and compositions for treating HCAP associated diseases, such as malignant and benign cell hyperproliferative diseases. Preferred diseases include EGFR associated diseases, including cancers, e.g., breast cancer.

### RELATED APPLICATIONS

This application claims the benefit of the filing date of U.S. Provisional Application Nos. 60/323,210, filed Sep. 18, 2001, and 60/332,350, filed Nov. 9, 2001, both of which are incorporated by reference herein in their entireties.

### BACKGROUND OF THE INVENTION

Normal tissue homeostasis is achieved by an intricate balance between the rate of cell proliferation and cell death. Disruption of this balance either by increasing the rate of cell proliferation or decreasing the rate of cell death can result in the abnormal growth of cells and is thought to be a major event in the development of cancer, as well as other cell proliferative disorders such as the restenosis that occurs after balloon angioplasty. A variety of proteins have been identified that are involved in regulating the balance between proliferation and death.

For example, the ErbB/HER family consists of four distinct members which are receptor tyrosine kinases, including the epidermal growth factor (EGF) receptor (EGFR or ErbB-1), ErbB-2, ErbB-3 and ErbB-4. These receptors are embedded in the plasma membrane and include four domains: an extracellular ligand binding domain, a transmembrane domain and an intracellular region, which includes a tyrosine kinase domain and a carboxy-terminal tail. ErbB-2 is the preferred heterodimeric partner of the other members of the family, ErbB-1, -3 and -4 (Graus-Porta et. al, 1997; Tzahar et. al, 1996), and its overexpression leads to oncogenic transformation (Hudziak et. al, 1987; Di Fiore et. al, 1987). In addition, co-expression of ErbB-2 together with ErbB-1 (Kokai et. Al, 1989), or one of the NRG receptors (Zhang et. al, 1996), exerts a synergistic effect on cell transformation. Accordingly, heterodimers with ErbB-2 generate a stronger proliferative signal compared with their respective homodimeric forms (Pinkas-Kramarski et. Al, 1996).

The members of the EGFR family play an essential role during growth and differentiation of many tissues. In addition, overexpression of these proteins is associated with several types of human cancers (reviewed in Hynes et al. (1994) Biochim Biophys Acta 1198(2-3), 165-84; Kim et al. (1999) Exp Cell Res 253(1), 78-87; and Hung et al. (1999) Seminars in Oncology 26 (4 Suppl 12), 51-9). The ErbB-2 proto-oncogene, which is also referred to as the Neu or Her-2 gene, is amplified and the protein overexpressed in 20-30% of human breast carcinomas, and this event correlates with poor prognosis. This observation strongly suggests that ErbB-2/Neu/Her-2 plays a direct role in the development of breast tumors. Furthermore, targeted expression of constitutively active Neu to the mouse mammary gland results in induction of multifocal mammary tumors in females harboring the transgene (Muller et al. (1988) Cell 54(1), 105-15; Andrechek et al.(2000) PNAS 97(7), 3444-9). In addition, Neu antisense treatment not only affects proliferation but also activates apoptotic pathways in Neu-overexpressing cells, demonstrating that this proto-oncogene is involved in both cell proliferation and cell survival (Roh et al. (2000) Cancer Res 60(3), 560-5).

Proteins involved in proliferation and cell death are often involved in a wide range of other cellular events and play a role in a variety of diseases. The functions of such proteins are often modulated by other interacting proteins, and these interacting proteins represent attractive targets for the design of agents to modulate cellular processes.

Thus, it would be desirable to identify proteins that modulate one or more proteins involved in cellular proliferation and death.

### SUMMARY OF THE INVENTION

In certain embodiments, the invention provides a method for treating an Hepatocellular Carcinoma Associated Protein (HCAP)-associated disease in a subject, comprising administering to the subject a pharmaceutically effective amount of an agent which inhibits the interaction between HCAP and an HCAP binding partner (HCAP-BP), such that the disease is treated in the subject. HCAP-BP can be a cell membrane receptor, such as a growth factor receptor, e.g., an epidermal growth factor receptor (EGFR). A preferred HCAP-BP is ErbB-2. The agent can be a small organic molecule, natural or synthetic, or any biological molecule. The agent can be a molecule or complex of molecules that mimics HCAP recognition site on HCAP-BP, or which mimics HCAP recognition site on HCAP-BP, e.g., ErbB-2.

In certain embodiments, the invention also provides a method for treating an HCAP associated disease in a subject, comprising administering to the subject a pharmaceutically effective amount of an agent which decreases the level and/or activity of HCAP in cells of a subject, such that the disease is treated in the subject. The agent can be a nucleic acid which decreases the level of HCAP RNA or protein, such as an HCAP specific ribozyme, an HCAP specific antisense molecule, an siRNA, or a nucleic acid which forms a triplex with the HCAP gene. The agent can also be a compound that prevents the recruitment of HCAP to the plasma membrane or a compound that induces the degradation of HCAP protein. The agent can also be a compound that decreases the membrane localization of an HCAP-BP that is a cell membrane receptor, such as ErbB-2.

An HCAP associated disease can be, e.g., a cell proliferative disease, such as a cell hyperproliferative disease. Examples of such diseases include cancer, such as an ErbB-2 associated cancer, e.g., breast cancer. The cell proliferative disease can also be a benign proliferative disease.

In certain embodiments, the invention also provides methods for inhibiting proliferation of a cell, comprising contacting the cell with an agent which inhibits the interaction between HCAP and an HCAP-BP or which decreases HCAP level or activity, such that the proliferation of the cell is inhibited. The cell can be in vitro, and can be obtained, e.g., from a subject, following which it is contacted ex vivo.

In certain embodiments, the invention provides isolated or recombinant polypeptide complexes comprising an HCAP polypeptide and an HCAP binding protein. In certain embodiments, the HCAP polypeptide comprises an amino acid sequence that is at least 85% identical to the amino acid sequence of SEQ ID No. 2, and optionally the HCAP polypeptide is at least 90%, at least 95%, at least 98% or at least 99% identical to the amino acid sequence of SEQ ID No.2. The HCAP polypeptide may also comprise a fragment of any of the foregoing that retains at least one function of a naturally occurring HCAP polypeptide, such as the ability to bind to an Erb/Her receptor family polypeptide, and optionally the fragment is at least 20, 30, 50, 100 or 200 amino acids in length. In certain embodiments the HCAP-BP is an Erb/Her receptor family polypeptide, such as an ErbB-1, ErbB-2, ErbB-3 or ErbB-4. Optionally the Erb/Her receptor family polypeptide is at least 85% identical to the amino acid sequence of SEQ ID No.4, and optionally at least 90%, at least 95%, at least 98% or at least 99% identical to the amino acid sequence of SEQ ID No. 4. The Erb/Her receptor family polypeptide may also comprise a fragment of any of the foregoing that retains at least one function of a naturally occurring Erb/Her receptor family polypeptide, such as the ability to catalyze protein phosphorylation or to bind to an HCAP polypeptide, and optionally the fragment is at least 20, 30, 50, 100 or 200 amino acids in length. In certain embodiments, an HCAP or Erb/Her receptor family polypeptide complex further comprises an additional moiety, such as an epitope tag, a purification moiety and a detection moiety. Optionally the HCAP or Erb/Her receptor family polypeptide is a fusion protein.

The invention further provides methods for identifying an agent which modulates the interaction between HCAP and an HCAP-BP, comprising contacting an isolated HCAP with an isolated HCAP-BP in the presence of a test agent under conditions in which, but for the presence of the test agent, HCAP interacts with HCAP-BP, such that a different level of: HCAP and HCAP-BP complex in the presence relative to the absence of the test agent indicates that the test agent modulates the interaction between HCAP and HCAP-BP. In vivo methods for identifying agents which modulate the interaction between HCAP and HCAP-BP are also contemplated, e.g., methods based on a “two-hybrid” system in eukaryotic cells, e.g., in mammalian cells.

The invention also provides kits comprising, e.g., an HCAP an HCAP-BP; a nucleic acid encoding HCAP, a nucleic acid encoding HCAP-BP, an HCAP, siRNA, an HCAP antisense molecule, or an HCAP ribozyme.

At least one of the advantages of the methods of the invention over known methods for treating hyperproliferative diseases, such as breast cancer, is that the methods of the invention can be applied in a tissue specific manner, thereby being less toxic than non-tissue specific mechanisms.

### BRIEF DESCRIPTION OF THE DRAWINGS

FIG. 1 A-C shows nucleotide (SEQ ID No.1) and amino acid (SEQ ID No.2) sequences of HCAP, as provided in GenBank Accession No. U92544.

FIG. 2 shows the co-immunoprecipitation of HCAP and Erb-B2 from cells.

FIG. 3A shows the resolution by denaturing gel electrophoresis of an immunoprecipitation of ErbB-2 complexes from T47D cells treated with Radicicol (RA) (3 μM) for 3 and 6 hours versus untreated cells. Band numbers 30 and 35 were identified by mass spectroscopic analysis as hepatocellular associated protein (HCAP). FIG. 3B shows an enlargement of a section of the gel.

FIG. 4 shows the effect of HCAP siRNA on the cellular localization of ErbB-2.

FIG. 5 A-B shows an mRNA nucleic acid sequence for ErbB-2 (SEQ ID No.3). FIG. 5C shows an amino acid sequence for ErbB-2 (SEQ ID No.4). Both sequences are as provided by Genbank accession no. X03363.

### DETAILED DESCRIPTION OF THE INVENTION

### 1. Overview

Certain aspects of the invention are based, in part, on the discovery that HCAP interacts with. ErbB-2. Certain aspects of the invention are based at least in part on the discovery that the interaction between HCAP and ErbB-2 can be disrupted by Radicicol in breast cancer cells. Radicicol is known to downregulate ErbB-2 levels at the cell surface and to inhibit cell proliferation, e.g., proliferation of cells which over-express ErbB-2 (see e.g., Chavany et al. (1996); Hartman et al. (1997) Int. J. Cancer 70:221; Miller et al. (1994) and Murakami et al. (1994). Accordingly, the invention provides methods for modulating cell proliferation, in particular, methods for inhibiting proliferation of hyperproliferative cells, such as cancer cells, e.g., breast cancer cells.

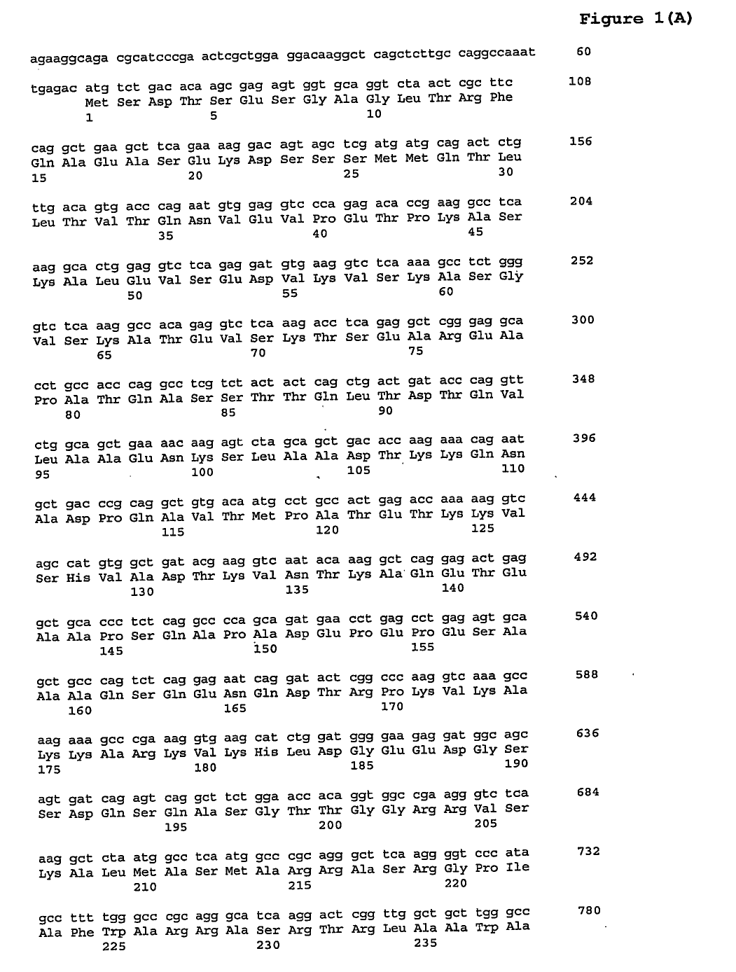

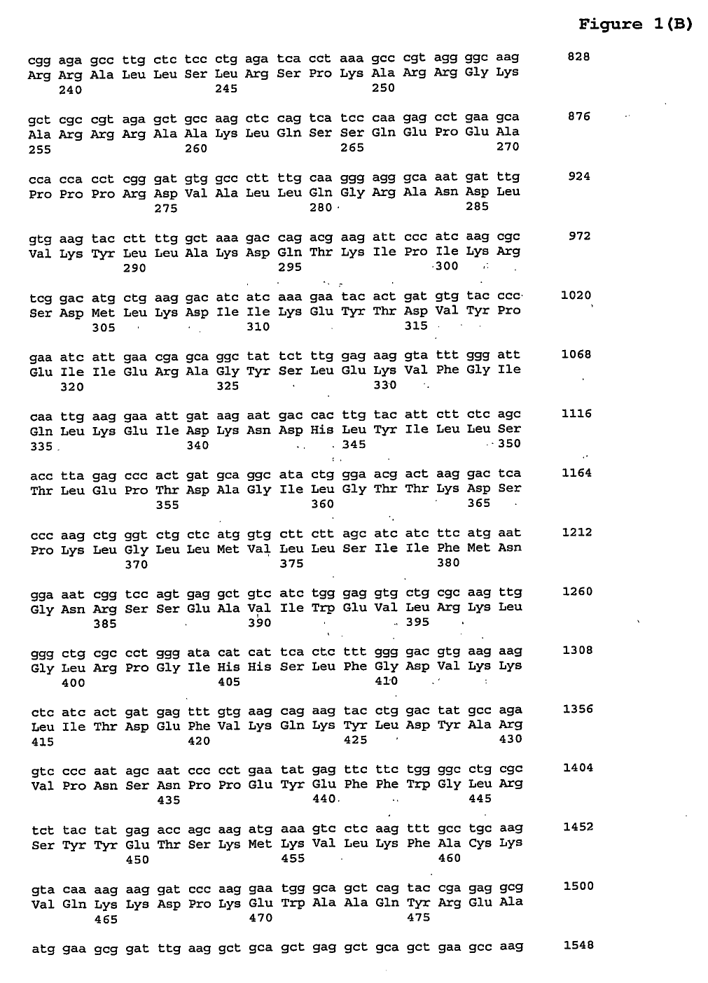

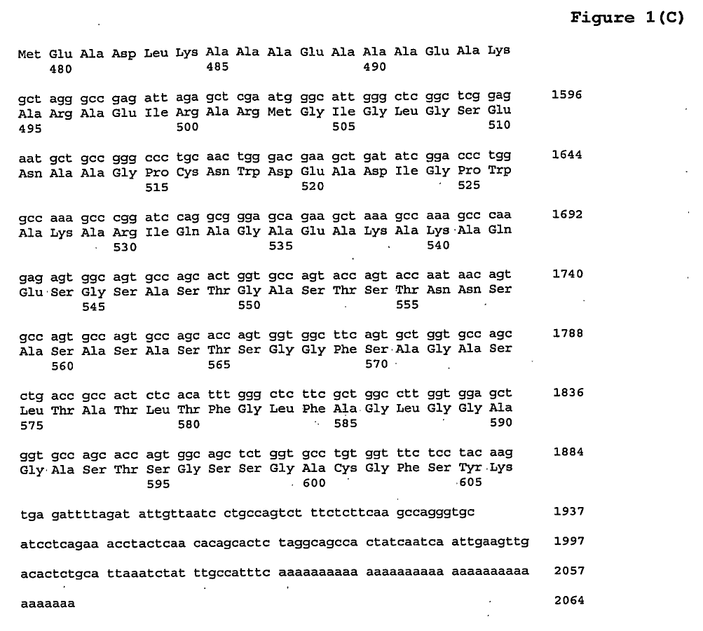

HCAP (Hepatocellular Carcinoma Associated Protein) is a protein which is also referred to as MAGE D2 and as Breast Cancer Associated Gene 1 (BCG1) (Lucas et al. (1999) Cancer Res. 59:4100). The nucleotide sequence of the complete cDNA of human HCAP is 2064 nucleotides long and is set forth in GenBank Accession No. U92544 and shown in FIG. 1 (SEQ ID NO: 1). The coding sequence corresponds to nucleotides 67 to 1887, and encodes a protein of 606 amino acids having the amino acid sequence set forth in GenBank Accession No. AAD00728 and shown in FIG. 1 (SEQ ID NO: 2). The region of homology with the other MAGE proteins is located in the central portion of the protein, at positions 276-478 (see Lucas et al., supra). It is homologous to the two hundred amino acids located at the COOH terminus of the other MAGE proteins.

The melanoma associated antigen “MAGE” family of proteins are recognized on many tumors by autologous cytotoxic T lymphocytes. MAGE A, B and C members are not expressed in normal cells, except for male germ-line cells, and should thus be tumor specific. HCAP is expressed in many, but not all, normal tissues (Lucas et al., supra). The intron-exon structure of HCAP is different from that of the other MAGE proteins. The members of the MAGE family are further described, e.g., in Chomez et al. (2001) Cancer Res. 61:5544.

### 2. Definitions

For convenience, certain terms employed in the specification, examples, and appended claims are collected here.

The terms “a” and “an” refer to “one or more” when used in this application, including the claims.

“Abnormal, growth of cells” means cell growth independent of normal regulatory mechanisms (e.g., loss of contact inhibition).

An “agent which inhibits the interaction between HCAP and an HCAP-BP” is an agent which interferes in the association between the two polypeptides. The inhibition can be, at least about 20%, preferably at least about 40%, even more preferably at least about. 50%, 70%, 80%, 90%, 95%, and most preferably at least about 98% of the interaction, between HCAP and an HCAP-BP. An agent can be, e.g., a small molecule, DNA, RNA, protein or variants thereof.

The term “antiproliferative” therapeutic or agent refers to an agent or therapeutic which inhibits, at least partly, cell proliferation.

“ErbB-2” which is referred to herein interchangeably as “ErbB-2”, “HER-2”, “Neu”, or “neu proto-oncogene”, encodes a p185 tumor antigen which is a growth factor receptor having extracellular, transmembrane, and intracellular domains. The oncogenic form of this protein (sometimes referred to as “oncogenic ErbB-2” or “c-ErbB-2”) contains a single amino acid point mutation located in the transmembrane domain, causing the receptor to become constitutively active, i.e., active in the absence of ligand. Over-expression of the normal receptor in a cell also causes the cell to become transformed.

The term “compound” refers generally to a molecule or complex of molecules. A compound can be an inorganic or organic molecule, e.g., a peptide, protein, nucleic acid. A compound is preferably a small molecule.

“Contacting a cell with an agent” refers to an action which results in the access of the agent to the cell, whether the agent is naked or is associated with other molecules, e.g., molecules facilitating the targeting of the agent to the cell or the entrance of the agent into a cell.

The term “cytostatic” when referring to the activity of an agent means that the agent causes the cell to enter cell cycle arrest without immediately killing the cell. Thus, removal of the drug from the environment of the cell results in the regain of cell proliferation.

A “disease associated with HCAP” or “HCAP associated disease” refers to a disease that can be treated by administering to a subject having such a disease an agent which modulates the interaction between HCAP and an HCAP binding polypeptide (HCAP-BP) or an agent which modulates HCAP levels or activity. A preferred disease associated with HCAP is a disease associated with a growth factor receptor, e.g., ErbB-2, such as a cancer associated with an excessive level of ErbB-2 protein or a mutated form of ErbB-2.

An “effective amount” of an agent of the invention, with respect to the subject method of treatment, refers to an amount of an agent of the invention in a preparation which, when applied as part of a desired dosage regimen brings about a change in the rate of cell proliferation and/or the state of differentiation of a cell so as to produce an amount of cell proliferation according to clinically acceptable standards for the disorder to be treated or the cosmetic-purpose.

An “EGFR associated disease” is a disease which is caused by or contributed to by excessive or insufficient EGFR stimulation and which may be caused by over-expression of an EGFR or a mutant form of the receptor. It may also be caused by the presence of an excess ligand for the receptor.

An “Erb/Her receptor family polypeptide” is any polypeptide of the Erb/Her receptor family, including ErbB-1, ErbB-2 (SEQ ID Nos. 3 and 4), ErbB-3, ErbB-4 and polypeptides that are at least 85% identical (optionally at least 90%, 95%, 98% or 99% identical) to any of the foregoing, as well as fragments that retain a functional activity, such as the ability to catalyze protein phosphorylation or the ability to bind to an HCAP polypeptide.

An “ErbB-2 associated disease” refers to a disease which is caused by or contributed to by excessive or insufficient ErbB-2 stimulation, resulting, e.g., from over-expression of ErbB-2 or a mutation in ErbB-2 or the presence of excess ligand for the receptor. An “ErbB-2 associated cancer” refers to a cancer which is caused by or contributed to by excessive ErbB-2 stimulation, resulting, e.g., from over-expression of ErbB-2 or a mutation in ErbB-2 or the presence of excess ligand for the receptor. Exemplary ErbB-2 associated cancers include carcinomas, e.g., breast carcinoma.

The terms “excessive cell proliferation,” used interchangeably herein with “hyper-proliferation” of cells refers to cells, which divide more often than their normal or wild-type counterpart. Thus, cells are excessively proliferating when they double in less than 24 hours if their normal counterparts double in 24 hours. Excessive proliferation can be detected by simple counting of the cells, with or without specific dyes, or by detecting DNA replication or transcription, such as by measuring incorporation of a labeled molecule or atom into DNA or RNA.

The term “HCAP polypeptide” refers to any polypeptide that is at least 85% identical (optionally at least 90%, 95%, 98% or 99% identical) to the amino acid sequence of SEQ ID No. 2 or a fragment thereof that retains the ability to interact with an HCAP-BP.

The term “including” is used herein to mean, and is used interchangeably with, the phrase “including but not limited to”.

“Inhibiting cell proliferation” refers to decreasing the rate of cell division, by interrupting or slowing down the cell cycle. The term refers to complete blockage of cell proliferation, i.e., cell cycle arrest, as well as to a lengthening of the cell cycle. For example, the period of a cell cycle can be increased by about 10%, about 20%, about 30, 40, 50, or 100%. The duration of the cell cycle can also be augmented by a factor of two, three, 4, 5, 10 or more.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject polypeptides preferably includes no more than 10 kilobases (kb) of nucleic acid sequence which naturally immediately flanks the subject gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “ligand” refers to an agent that binds at the receptor site. “Modulating cell differentiation” refers to the stimulation or inhibition of cell differentiation.

“Normalizing cell proliferation” refers to reducing the rate of cell proliferation of a cell that proliferates excessively relative to that of its normal counterpart, or increasing the rate of cell proliferation of a cell that proliferates poorly relative to its normal or wild-type counterpart, such that its rate of proliferation is similar to that in the normal counterpart cell.

As used herein, the term “nucleic acid” refers to polynucleotides or oligonucleotides such as ribonucleic acid (RNA), and, where appropriate, deoxyribonucleic acid (DNA). The term should also be understood to include, as equivalents, analogs of either RNA or DNA made from nucleotide analogs and as applicable to the embodiment being described, single (sense or antisense) and double-stranded polynucleotides.

The term “or” is used herein to mean, and is used interchangeably with, the term “and/or”, unless context clearly indicates otherwise.

A “patient” or “subject” to be treated by the subject method can mean either a human or non-human animal, and preferably a mammal, e.g., a bovine, ovine, porcine, non-human primate, canine, or feline.

The terms “polypeptide” and “protein” are used interchangeably herein.

The term “proliferative disorder” refers to any disease/disorder of a tissue marked by unwanted or aberrant proliferation of at least some cells in the tissue. Such diseases include cancer, as well as benign diseases or disorders, such as warts or other benign tumors.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins particularly other proteins which may substantially mask, diminish, confuse or alter the characteristics of the component proteins either as purified preparations or in their function in the subject. reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein. A “recombinant protein complex” or “recombinant complex” is a complex comprising one or more recombinant proteins.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention.

The term “transformed cell” refers to a cell which was converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. Transformed cells include cancer cells, such as cells over-expressing a proto-oncogene or expressing a mutated form of an proto-oncogene (i.e., an oncogene). Transformed cells also include cells infected by a microorgarnism, e.g., viruses. Exemplary viruses are retroviruses.

“Treating” a disease refers to preventing, curing or improving at least one symptom of a disease.

### 3. Exemplar Methods and Therapeutic Agents

In certain embodiments, the invention provides methods for modulating the interaction between HCAP and an HCAP-BP and methods for modulating HCAP levels or activity. In a preferred embodiment, the HCAP-BP is a growth factor receptor, such as ErbB-2.

In certain embodiments, the invention provides methods for modulating proliferation and/or differentiation of a cell by modulating the level of or activation state of an HCAP. In certain preferred embodiment, the invention provides methods for decreasing cellular proliferation by inhibiting HCAP. In a particularly preferred embodiment, the invention provides methods for treating cancers or other hyperproliferative diseases characterized by Erb-B2 expression in all or a subset of the cancerous or hyperproliferative cells. In an exemplary embodiment, the invention provides methods for treating breast cancers, and particularly taxol-resistant breast cancers, by administering an HCAP siRNA nucleic acid.

In one embodiment, proliferation of a cell is inhibited by contacting the cell with an agent which inhibits the interaction between HCAP and an HCAP-BP. The agent can be a small molecule, which may, e.g., interact with an HCAP-BP recognition site on HCAP. The agent can also be a peptide, e.g., a portion of HCAP or HCAP-BP, which portion mediates the interaction between the two polypeptides. Such agents can be identified according to methods described herein. Assays using cell lines can be conducted to confirm that the agents inhibit cell proliferation.

In another embodiment, proliferation of a cell is inhibited by contacting the cell with an agent which decreases the HCAP protein level or activity in a cell. The agent can be an agent which interacts with the HCAP genomic DNA, RNA or protein. The following are exemplary agents that can be used.

(i) Antisense Nucleic Acids

One method for decreasing the level of HCAP in a cell comprises introducing into the cell antisense molecules which are complementary to at least a portion of HCAP RNA. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably: oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652: PCT Publication No. WO 88/09810, published Dec. 15, 1988), hybridization-triggered cleavage agents (see, e.g., Krol et al., 1988, BioTechniques 6: 958-976) or intercalating agents (see, e.g., Zon, 1988, Pham. Res. 5: 539-549).

In a preferred aspect of the invention, an antisense oligonucleotide is provided, preferably as single-stranded DNA. The oligonucleotide may be modified at any position on its structure with constituents generally known in the art. For example, the antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylnethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent transport agent, hybridization-triggered cleavage agent, etc. An antisense molecule can be a “peptide nucleic acid” (PNA). PNA refers to an antisense molecule or anti-gene agent which comprises an oligonucleotide of at least about 5 nucleotides in length linked to a peptide backbone of amino acid residues, optionally ending in lysine. The terminal lysine confers improved solubility to the composition. PNAs preferentially bind complementary single stranded DNA or RNA and stop transcript elongation, and may be modified with a polyethylene glycol moiety (“pegylated”) to extend their lifespan in the cell.

The antisense nucleic acids of the invention comprise a sequence complementary to at least a portion of a target RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

The synthesized antisense oligonucleotides can then be administered to a cell in a controlled manner. For example, the antisense oligonucleotides can be placed in the growth environment of the cell at controlled levels where they may be taken up by the cell. The uptake of the antisense oligonucleotides can be assisted by use of methods well known in the art.

In an alternative embodiment, the antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc.

Antisense therapy for a variety of cancers is in clinical phase and has been discussed extensively in the literature. Reed reviewed antisense therapy directed at the Bcl-2 gene in tumors; gene transfer-mediated overexpression of Bcl-2 in tumor cell lines conferred resistance to many types of cancer drugs. (Reed, J. C., N.C.I. (1997) 89:988-990). The potential for clinical development of antisense inhibitors of ras is discussed by -Cowsert, L. M., Anti-Cancer Drug Design (1997) 12:359-371. Additional important antisense targets include leukemia (Geurtz, A. M., Anti-Cancer Drug Design (1997) 12:341-358); human C-ref kinase (Monia, B. P., Anti-Cancer Drug Design (1997) 12:327-339); and protein kinase C (McGraw et al., Anti-Cancer Drug Design (1997) 12:315-326.

(ii) Ribozymes

In another embodiment, the level of HCAP in a cell can be reduced by introduction of a ribozyme into the cell or nucleic acid encoding such. Ribozyme molecules designed to catalytically cleave mRNA transcripts can also be introduced into, or expressed, in cells to inhibit expression of a target gene (see, e.g., Sarver et al., 1990, Science 247:1222-1225 and U.S. Pat. No. 5,093,246). One commonly used ribozyme motif is the hammerhead, for which the substrate sequence requirements are minimal. Design of the hammerhead ribozyme is disclosed in Usman et al., Current Opin. Struct. Biol. (1996) 6:527-533. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16-17; Ojwang et al., Proc. Natl. Acad. Sci. (USA) (1992) 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059-7071. Preparation and use of ribozyme fragments in a hammerhead structure are also described by Koizumi et al., Nucleic Acids Res. (1989) 17:7059-7071. Preparation and use of ribozyme fragments in a hairpin structure are described by Chowrira and Burke, Nucleic Acids Res. (1992) 20:2835. Ribozymes can also be made by rolling transcription as described in Daubendiek and Kool, Nat. Biotechnol. (1997) 15(3):273-277.

The mechanism of ribozyme action involves sequence specific hybridization of the ribozyme molecule to complementary target RNA, followed by an endonucleolytic cleavage event. The composition of ribozyme molecules preferably includes one or more sequences complementary to the target gene mRNA, and the well known catalytic sequence responsible for mRNA cleavage or a functionally equivalent sequence (see, e.g., U.S. Pat. No. 5,093,246.

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. W089/05852. Hammerhead ribozyme sequences can be embedded in a stable RNA such as a transfer RNA (tRNA) to increase cleavage efficiency in vivo (Periman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes preferably contain a hybridizing region complementary to two regions, each of at least 5 and preferably each 6, 7, 8, 9, 10, 11, 12, 13, 14, 15, 16, 17, 18, 19 or 20 contiguous nucleotides in length of the target mRNA.

As in antisense approaches which are also known in the art, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous target messages. Because ribozymes unlike antisense molecules, are catalytic, a lower intracellular concentration is required for efficiency. Ribozymes can also be prepared in vitro and administered to a cell or subject.

To design ribozymes, computer generated predictions of secondary structure can used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (seeMilner et al. (1997) Nat Biotechnol 15: 537-41; and Patzel and Sczakiel (1998) Nat Biotechnol 16: 64-8). Additionally, U.S. Pat. No. 6,251,588 describes methods for evaluating oligonucleotide probe sequences so as to predict the potential for hybridization to a target nucleic acid sequence.

Warashina et al. ((2001) PNAS USA 98: 5572-77) have described improved ribozyme compositions that includes a constitutive transport element (CTE) which recruits RNA helicase (Tang et al; (1997) Science 276: 1412-5; Gruter et al. (1998) Mol Cell 1: 649-59; Braun et al. (199) EMBO J 18: 1953-65; Hodge et al. (1999) EMBO J 18: 5778-88; Kang et al. (1999) Genes Dev. 13: 1126-39); Li et al. (1999) PNAS USA 96: 709-14; Schmitt et al. (1999) EMBO J 18: 4332-47 and Tang et al. (2000) J Biol Chem 275: 32694-32700).

Further compositions, methods and applications of ribozyme technology are provided in U.S. patent application Ser. Nos. 6,281,375, 6,277,565, 6,274,342, 6,274,339, 6,271,440, and 6,271,436.

(iii) siRNAs

Another method for decreasing or blocking gene expression is by introducing double stranded small interfering RNAs (siRNAs), which mediate sequence specific mRNA degradation. RNA interference (RNAi) is the process of sequence-specific, post-transcriptional gene silencing in animals and plants, initiated by double-stranded RNA (siRNA) that is homologous in sequence to the silenced gene. In vivo, long siRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. Nature 2001 ;411(6836):494-8). As described in the Examples below, the administration of an siRNA targeted to HCAP causes mislocalization of ErbB-2 in a breast cancer cell line.

RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashir et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discemable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phophoramidites and thymidine phosphoramidite (Proligo, Germany). Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allow selection of those sequences likely to occur in exposed single stranded regions of a folded mRNA. Methods and compositions for designing appropriate oligonucleotides may be found, for example, in U.S. Pat. No. 6,251,588. Messenger RNA (mRNA) is generally thought of as a linear molecule which contains the information for directing protein synthesis within the sequence of ribonucleotides, however studies have revealed a number of secondary and tertiary structures exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art- e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the targeted gene product following sufficient time for turnover of the endogenous pool after new protein synthesis is repressed, and Northern blot analysis to determine the level of existing target mRNA.

Further compositions, methods and applications of RNAi technology are provided in U.S. patent application Ser. Nos. 6,278,039, 5,723,750 and 5,244,805.

(iv). Triplex Formation

Gene expression can be reduced by targeting deoxyribonucleotide sequences complementary to the regulatory region of the target gene (i.e., the gene promoter and/or enhancers) to form triple helical structures that prevent transcription of the gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

(v) Dominant Negative Mutants

In another embodiment, a dominant negative mutant of HCAP is used to compete with the action of wild-type HCAP in a cell. An HCAP dominant negative mutant can be a polypeptide which interacts with an HCAP-BP, but which fails to exert a biological effect. Preferably, a dominant negative mutant polypeptide will be overproduced. Dominant negative mutants can be prepared, e.g., by making point mutations or by fusing different polypeptides of various lengths to the terminus of a protein. General strategies are available for making dominant negative mutants. See Herskowitz, Nature (1987) 329:219-222.

(vi) Use of Agents Inhibiting Transcription of an HCAP Gene

In another embodiment, cell proliferation is inhibited by contacting a cell with an agent which decreases the expression of a gene encoding HCAP. Such agents can be identified as described herein and also according to methods known in the art.

In other embodiments of the invention, the activity of HCAP in a cell is inhibited by preventing the recruitment of HCAP to the cell surface membrane.

In another embodiment, the invention provides a method for stimulating cell proliferation. In one embodiment, cell proliferation is stimulated by contacting a cell with an agent that stimulates the interaction between HCAP and an HCAP-BP, e.g., ErbB-2. Such compounds can be obtained as further described herein. In another embodiment, cell proliferation is stimulated by increasing in a cell the HCAP protein or activity level. For example, a cell can be transfected with a nucleic acid encoding an HCAP protein or portion thereof Nucleic acids encoding HCAP can be obtained according to methods known in the art and using the published nucleotide sequence of the gene, e.g., human gene encoding HCAP. Expression vectors are also well known in the art.

### 4. Preparation of Nucleic Acids and Polypeptides

Nucleic acids for use according to the methods disclosed herein, e.g., for administration to a subject, can be prepared by methods known in the art. A nucleic acid encoding an HCAP, e.g., human HCAP can be obtained by, e.g., reverse transcription-polymerase chain reaction (RT-PCR), by screening nucleic acid libraries or from publicly available DNA clones. The nucleotide and amino acid sequences of human HCAP are set forth in FIG. 1. It may not be necessary to express the full length polypeptide in a cell of a subject, and a portion thereof sufficient for binding to an HCAP-BP may be sufficient. The portion of HCAP that is sufficient for binding to an HCAP-BP can be determined by expressing various portions of HCAP and determining in in vitro or in cell assays which portion is sufficient for interacting with HCAP. Such methods, are known in the art. Nucleic acids encoding an HCAP-BP, e.g., ErbB-2 can be obtained by RT-PCR or they may be publicly available. A nucleotide sequence encoding human ErbB-2 (SEQ ID No.3), differing from human EGFR, and encoded amino acid sequence (SEQ ID No.4) are provided in GenBank under Accession No. X03363.

Certain amino acid deletions, additions and substitutions are permitted, provided that the polypeptide retains its capability to bind to its partner. For example, it is expected that polypeptides having conservative amino acid substitutions will have the same activity as the wild-type polypeptide. Polypeptides which are shorter or longer than HCAP or HCAP-BP or which contain from one to 20 amino acid deletions, insertions or substitutions and which have a biological activity that is essentially identical to that of HCAP or HCAP-BP are referred to herein as equivalents of HCAP or HCAP-BP, respectively. Equivalent polypeptides also include polypeptides having an amino acid sequence which is at least 80%, preferably at least about 90%, even more preferably at least about 95% and most preferably at least 98% identical or similar to that of the wild-type polypeptide.

Any means for the introduction of polynucleotides, e.g., encoding HCAP, or a ribozyme or dsRNA for RNAi or antisense molecule, into cells in vitro or in mammals, human or non-human, may be adapted to the practice of this invention for the delivery of the various constructs of the invention into the intended cell or recipient. In one embodiment of the invention, DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject, e.g., to breast tumors.

In a preferred method of the invention, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans. The following additional guidance on the choice and use of viral vectors may be helpful to the practitioner. Such embodiments of the subject expression constructs are specifically contemplated for use in various in vivo and ex vivo gene therapy protocols.

Tissue specific targeting and/or expression of a construct of the invention can be achieved by using a tissue specific promoter. For example, to obtain breast tumor cell specific expression, a promoter that is expressed essentially specifically to these cells can operably linked to a nucleic acid encoding the desired RNA or protein. The targeting vehicle, e.g., liposome or viral vector can also be modified to contain on its surface molecules that will target it to the target cells.

Polypeptides for use in the invention can be synthesized in prokaryotes or eukaryotes or cells thereof and purified according to methods known in the art. For example, recombinant polypeptides can be synthesized in human cells, mouse cells, rat cells, insect cells, yeast cells, and plant cells. Polypeptides can also be synthesized in cell free extracts, e.g., reticulocyte lysates or wheat germ extracts. Purification of proteins can be done by various methods, e.g., chromatographic methods (see, e.g., Robert K Scopes “Protein Purification: Principles and Practice” Third Ed. Springer-Verlag, N.Y. 1994). In one embodiment, the polypeptide is produced as a fusion polypeptide comprising an epitope tag, e.g., consisting of about six consecutive histidine residues. The fusion polypeptide can then be purified on a Ni⁺⁺ column. By inserting a protease site between the tag and the polypeptide, the tag can be removed after purification of the peptide on the Ni⁺⁺ column. These methods are well known in the art and commercial vectors and affinity matrices are commercially available.

Administration of polypeptides can be done by mixing them with liposomes, as described above. The surface of the liposomes can be modified by adding molecules that will target the liposome to the desired physiological location.

In one embodiment, a polypeptide is modified so that its rate of traversing the cellular membrane is increased. For example, a polypeptide can be fused to a second peptide which promotes “transcytosis,” e.g., uptake of the peptide by cells. In one embodiment, the peptide is a portion of the HIV transactivator (TAT) protein, such as the fragment corresponding to residues 37-62 or 48-60 of TAT, portions which are rapidly taken up by cell in vitro (Green and Loewenstein, (1989) Cell 55:1179-1188). In another embodiment, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides can be fused to a peptide consisting of about amino acids 42-58 of Drosophila antennapedia or shorter fragments for transcytosis. See for example, Derossi et al. (1996) J Biol Chem 271:18188-18193; Derossi et al. (1994) J Biol Chem 269:10444-10450; and Perez et al. (1992) J Cell Sci 102:717-722.

### 5. Polypeptide Complexes

In an additional aspect, the invention provides complexes comprising an HCAP polypeptide and an HCAP-AP. In one embodiment, the invention provides an isolated or recombinant protein complex comprising an HCAP polypeptide in combination with a cell membrane receptor, such as an EGFR. Exemplary complexes include isolated or recombinant complexes comprising an HCAP polypeptide and an ErbB-1 polypeptide, ErbB-2 polypeptide, ErbB-3 polypeptide or ErbB-4 polypeptide. In certain embodiments, the complex comprises a soluble portion of a cell membrane receptor, such as the cytoplasmic portion, and an HCAP polypeptide. In certain embodiments, the complex comprises a cell membrane receptor polypeptide including a transmembrane domain, in which case it may be desirable to include a solubilizing agent (such as a detergent, preferably a non-ionic or zwitterionic detergent; exemplary detergents include Triton X-100 and octylglucoside) or a micelle-forming agent, such as one or phospholipids. In certain embodiments, the complex comprises an HCAP polypeptide that comprises a fragment of an HCAP protein that is sufficient for binding to the HCAP-AP. In certain embodiments, the complex comprises an HCAP-AP polypeptide that comprises a fragment of an HCAP-AP protein that is sufficient for binding to HCAP. In certain embodiments, the complex comprises an oncogenic mutant of an HCAP-AP. For example, a complex may comprise an oncogenic form of an ErbB-2 polypeptide.

Complexes of the invention may be generated in a variety of ways. For example, a complex may be isolated from a cell expressing the appropriate proteins (e.g. a breast tumor cell line) by immunoprecipitation with an antibody directed to one of the members of the complex. In another example, a complex may be prepared by expressing the polypeptides of the complex in a recombinant cell (e.g. an Escherichia coli cell, a Saccharomyces cerevisiae cell, an insect cell that is compatible with baculovirus expression vectors, a Chinese hamster ovary cell, a breast cancer cell line, etc.). The recombinant proteins may then be combined in vitro to reconstitute the complex. In another example, a complex may be formed by attaching one member of the complex to an insoluble matrix (e.g. an agarose, polyacrylamide, Sepharose or other bead or resin), optionally poured to form a column, and passing a solution comprising a second member of the complex (e.g. purified protein or crude cellular extract) over the matrix, thereby reconstituting a matrix-bound complex. In certain embodiments, one or more of the proteins in a complex comprise an additional moiety, such as an additional polypeptide sequence or other added compound, with a particular function. Examples of such moieties include epitope tags that facilitate detection of the recombinant polypeptide with an antibody, a purification moiety that facilitates purification (e.g. by affinity purification), or a detection moiety, that facilitates detection of the polypeptide in vivo or in vitro. Often, a single moiety will provide multiple functionalities. For example, an epitope tag will generally also assist in purification, because an antibody that recognizes the epitope can be used in an affinity purification procedure as well. Examples of commonly used epitope tags are: a hemaglutinin (HA) tag, a hexahistidine tag, a V5 tag, a Glu-Glu tag, a c-myc tag, a vesicular stomatits virus G protein (VSV-G) tag, a FLAG tag, an enterokinase cleavage site tag and a T7 tag. Commonly used purification moieties include: a hexahistidine tag, a glutathione-S-transferase domain, a cellulose binding domain and a biotin tag. Commonly used detection moieties include fluorescent proteins (e.g. green fluorescent proteins), a biotin tag, and chromogenic/fluorogenic enzymes (e.g. beta-galactosidase and luciferase). Commonly used antigenic moieties include the keyhole limpet hemocyanin and serum albumins. Note that these moieties need not be polypeptides and need not be connected to the polypeptide by a traditional peptide bond. When the additional moiety is a polypeptide that is fused, by a normal polypeptide bond to the polypeptide of the complex, the polypeptide of the complex may be termed a fusion protein.

### 6. Identification of Agents which Modulate HCAP Level or Activity or its Interaction with an HCAP-BP

In one embodiment, an agent which modulates the expression of the HCAP gene is identified by contacting cells expressing an HCAP gene with-test agents, and monitoring the level of expression of the HCAP gene. Alternatively, agents which modulate the expression of an HCAP gene can be identified by conducting assays using the promoter region of the HCAP gene and screening for compounds which modify binding of proteins to the promoter region. The promoter region of an HCAP gene can be isolated, e.g., by screening a genomic library with a probe corresponding to an HCAP gene. Such methods are known in the art.

One exemplary screening assay of the present invention includes the steps of contacting HCAP or functional fragment thereof or a binding partner with a test agent or library of test agents and detecting the formation of complexes. For detection purposes, the molecule can be labeled with a specific marker and the test agent or library of test agents labeled with a different marker. Interaction of a test agent with HCAP or fragment thereof or binding partner can then be detected by determining the level of the two labels after an incubation step and a washing step. The presence of two labels after the washing step is indicative of an interaction.

Inhibitors of HCAP can also be agents that bind to HCAP, and thereby prevent it from functioning normally, or that degrade or cause HCAP to be degraded. For example, such an agent can be an antibody or derivative thereof which interacts specifically with HCAP. Preferred antibodies are monoclonal antibodies, humanized antibodies, human antibodies, and single chain antibodies. Such antibodies can be prepared and tested as known in the art.

Agents that modulate, e.g., inhibit or stimulate, the interaction between HCAP and an HCAP-BP, e.g., ErbB-2, can be identified in cell free assays or in cell assays. In a preferred embodiment, cell-free assays for identifying such agents comprise forming a reaction mixture containing HCAP and a binding partner, i.e., an HCAP-BP in the presence or absence of a test agent or a library of test agents and then measuring any increase or decrease in the association between the HCAP and the HCAP-BP. A preferred binding partner is a growth factor receptor, e.g., ErbB-2, or portions thereof sufficient for interacting with HCAP. A test agent can be, e.g., a derivative of an HCAP-BP, e.g., a biologically inactive peptide, or a small molecule.

Another exemplary screening assay of the present invention includes the steps of (a) forming a reaction mixture including: (i) HCAP, (ii) a binding partner (e.g., ErbB-2), and (iii) a test agent; and (b) detecting interaction of HCAP and the binding partner. HCAP and the binding partner can be produced recombinantly, purified from a source, e.g., plasma, or chemically synthesized, as described herein. A statistically significant change (potentiation or inhibition) in the interaction of HCAP and a binding partner in the presence of the test agent, relative to the interaction in the absence of the test agent, indicates a potential agonist (mimetic or potentiator) or antagonist (inhibitor) of HCAP bioactivity for the test agent. The agents of this assay can be contacted simultaneously. Alternatively, HCAP can first be contacted with a test agent for an appropriate amount of time, following which the binding partner is added to the reaction mixture. The efficacy of the agent can be assessed by generating dose response curves from data obtained using various concentrations of the test agent. Moreover, a control assay can also be performed to provide a baseline for comparison. In the control assay, isolated and purified HCAP or binding partner is added to a composition containing the binding partner or HCAP, and the formation of a complex is quantitated in the absence of the test agent.

Complex formation between HCAP and a binding partner may be detected by a variety of techniques. Modulation of the formation of complexes can be quantitated using, for example, detectably labeled proteins such as radiolabeled, fluorescently labeled, or enzymatically labeled HCAP or binding partners, by immunoassay, or by chromatographic detection. An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test agents can be immobilized on a sensor surface, e.g., which forms one wall of a micro-flow cell. A solution containing the HCAP, functional fragment thereof, HCAP analog or binding partner is then flown continuously over the sensor surface. A change in the resonance angle as shown on a signal recording, indicates that an interaction has occurred. This technique is further described, e.g., in BIAtechnology Handbook by Pharmacia.

Typically, it will be desirable to immobilize either HCAP or its binding partner to facilitate separation of complexes from uncomplexed forms of one or both of the proteins, as well as to accommodate automation of the assay. Binding of HCAP to a binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/HCAP (GST/HCAP) fusion proteins can be adsorbed onto glutathione sepharose beads (Sigma Chemical, St. Louis, Mo.) or glutathione derivatized microtitre plates, which are then combined with the binding partner, e.g. an ³⁵S-labeled binding partner, and the test agent, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of HCAP or binding partner found in the bead fraction quantitated from the gel using standard electrophoretic techniques such as described in the appended examples.

Other techniques for immobilizing proteins on matrices are also available for use in the subject assay. For instance, either HCAP or its cognate binding partner can be immobilized utilizing conjugation of biotin and streptavidin. For instance, biotinylated HCAP molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with HCAP can be derivatized to the wells of the plate, and HCAP trapped in the wells by antibody conjugation. As above, preparations of a binding partner and a test agent are incubated in the HCAP presenting wells of the plate, and the amount of complex trapped in the well can be quantitated. Exemplary methods for detecting such complexes, in addition to those described above for the GST-immobilized complexes, include immunodetection of complexes using antibodies reactive with the binding partner, or which are reactive with HCAP and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the binding partner. To illustrate, the binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes that rely on immunodetection for quantitating one of the proteins trapped in the complex, antibodies against the protein, such as anti-HCAP antibodies, can be used. Alternatively, the protein to be detected in the complex can be “epitope tagged” in the form of a fusion protein which includes, in addition to the HCAP sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

In yet another embodiment, HCAP and HCAP-BP can be used to generate an interaction trap assay (see also, U.S. Pat. No.: 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene that comprises the coding sequence for a DNA-binding domain of a transcriptional activator can be fused in frame to the coding sequence for a “bait” protein, e.g., an HCAP polypeptide of sufficient length to bind to HCAP-BP. The second hybrid protein encodes a transcriptional activation domain fused in frame to a gene encoding a “fish” protein, e.g., an HCAP-BP of sufficient length to interact with the HCAP polypeptide portion of the bait fusion protein. If the bait and fish proteins are able to interact, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, preferably a yeast cell, e.g., S cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector. Interaction trap assays may also be performed in mammalian and bacterial cell types.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as an HCAP sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, 1cI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication W094/10300).

In preferred embodiments, any enzymatic activity associated with the bait or fish proteins is inactivated.

The interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of an HCAP-HCAP-BP complex results in a detectable signal produced by the expression of the reporter gene. Accordingly, the level of formation of a complex in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

### 7. Assays for Determining the Effect of Agents on Cell Proliferation and Differentiation

The effect of an agent on cell proliferation can be determined, e.g., by incubating cells with varying amounts of the agents and counting the cells over time. Viable cells can be counted by staining the cells with a specific dye, e.g., Trypan Blue, according to methods well known in the art. Other methods include measuring the incorporation of a labeled molecule into DNA or RNA or protein of cells. For example, cell proliferation is often measured by ³H thymidine or 5-bromodeoxyuridine incorporation assays, also well known in the art. An increase in ³H thymidine or 5-bromodeoxyuridine incorporation in cells incubated with a test agent that is similar to that in cells non incubated with the test agent indicates that the test agent is essentially not inhibiting the proliferation of the cells. On the contrary, a lower ³H thymidine or 5-bromodeoxyuridine incorporation in cells incubated with a test agent relative to cells that were not treated with the test agent indicates that the test agent inhibits cell proliferation.

The effect of an agent on cell differentiation can be determined by visualization of the cells after having been contacted with the agent, preferably by comparison with cells which have not been contacted with the agent. The differentiation of certain cells is visible by the naked eye (e.g., that of 3T3L1 cells), whereas that of other cells may require the use of a microscope. Specific dyes can also be used to evaluate the state of differentiation of cells. Cell differentiation can also be monitored by measuring the expression level of certain genes, whose expression is known to vary during differentiation of the cells.

Such assays can be conducted on cells that are transformed, e.g., by ErbB-2. These can be cell lines or primary cell cultures. Numerous cell lines that are transformed by over-expression of a proto-oncogene or the presence of an oncogene are available, e.g., from the American Type Culture Collection (ATCC). Cell lines over-expressing a gene, e.g., a proto-oncogene can be prepared by transient, or preferably, stable transfection of cells with an expression plasmid containing the gene. Transfection methods are well known in the art and are also described in the examples. Nucleic acids for transforming cells, e.g., proto-oncogenes are also available in the art. Cell lines can also be obtained from transgenic animals expressing an oncogene. For example, MG 1361 is a breast carcinoma cell line obtained from the MMTV-neu transgenic mouse (Sacco et al., Breast Cancer Res. Treat., 47:171-180 (1998)). Primary cell cultures can be established from biopsies obtained from subjects. For example, primary tissue cultures of cells over-expressing an activated form of Neu can be prepared from biopsies of subjects having breast cancer.

The effect of the agents on the invention on cell proliferation, and in particular on malignant cell proliferation, can be determined by using animal models. For example, transgenic mice can be produced that express ErbB-2 or other proto-oncogene or oncogene under the control of a promoter, e.g., a tissue specific promoter. Such mice develop carcinomas that have genetic and pathological features that closely resemble human cancers. For example, mice expressing viral polyorna middle T antigen under the control of the MMTV promoter produces highly metastatic mammary tumors (Guy et al. (1994) Genes and Dev. 8:23). Nude mice in which tumor cell lines have been administered can also be used. For example, breast cancer cell lines over-expressing ErbB-2 can be administered to nude mice, in a manner similar to that described with cell lines over-expressing c-src described in Biscardi et al. (1998) Mol. Carcinog. 21: 261). The ability of an agent to inhibit tumor formation or growth is then ascertained. In one embodiment, the size of the tumor is monitored by determining the tumor size and/or weight. The agents can be administered by a variety of ways including orally, subcutaneously, or intraperitoneally. Generally, at least two groups of animals are used in the assay, with at least one group being a control group which is administered the administration vehicle without the agent.

### 8. Diseases

The invention provides methods for treating HCAP associated diseases. In one embodiment, the invention provides methods for decreasing cell proliferation. The methods can be used, e.g., for inhibiting excessive cell proliferation, such as malignant or benign cell proliferation. In a preferred embodiment, the methods comprise administering to a subject in need thereof a cell proliferation-inhibitory amount of an agent which modulates the interaction between HCAP and HCAP-BP, e.g., ErbB-2, or an agent which modulates HCAP levels or activity.

The methods of the invention can be used to treat EriB-2 associated proliferative diseases, e.g., cancers. For example, amplification and/or over-expression of human erbB-2 gene, has been shown to correlate with a poor prognosis in breast and ovarian cancers, in particular, carcinomas (see, e.g., Slamon et al., Science 235:177-82 (1987); Slamon et al., Science 244:707-12 (1989)). Over-expression of erbB-2 has also been correlated with other carcinomas including carcinomas of the stomach, endometrium, salivary gland, lung, kidney, colon and bladder. Taxol-resistant breast cancers are often ErbB-2 positive.

In breast cancer, the basement membrane-encapsulated carcinoma in situ is a probable precursor of infiltrating ductal cancer (IDC), a lesion where local invasion occurs. There are two major types of carcinoma in situ in the breast: lobular carcinoma in situ (LCIS) and ductal carcinoma in situ (DCIS). LCIS represents a risk factor and it exhibits precursor activity (Sandgren et. Al, 1995), whereas DCIS more often develops into invasive carcinoma. DCIS development involves proliferation of malignant epithelial cells within the ducts and lobules of the breast, without invasion through the basement membrane (Kinzler et. Al, 1996; Fisher et. Al, 1996). As a result of the formation of micro-calcifications in tumor areas, DCIS can be detected by mammography and it currently represents 30% of all detected breast malignancies (Emster et. Al, 1996). Pathologists generally divide DCIS into five architectural subtypes (papillary, micropapillary, cribriform, solid, and comedo), often grouping the first four together as non-comedo. Comedo DCIS is generally associated with more aggressive clinical behavior (Lagios et. Al, 1996) and a set of landmarks, which include high nuclear grade, aneuploidy (Aasmundstand et. Al, 1990), higher proliferation rate, and overexpression of ErbB-2, which in the majority of cases is due to gene amplification (Slamon et. Al, 1987).

Overexpression of ErbB-2 in IDC is associated with a large component of poorly differentiated comedo-type DCIS (Barnes et. Al, 1991). Since comedo-type DCIS is often hormonally independent, the high proliferation rate and protection from apoptosis may be contributed by ErbB-2. Breast epithelial cells overexpressing ErbB-2 have significantly higher proliferation rates, and down-regulation of ErbB-2 levels causes inhibition of cellular proliferation or apoptosis. Reduction of ErbB-2 levels in an ovarian cancer cell line, by utilizing an ErbB-2 specific ribozyme, inhibited tumor growth in nude mice (Juhl et. Al, 1997). Further support for the involvement of ErbB-2 in the initiation and progression of breast cancer comes from the generation and analysis of transgenic mice. In several transgenic models, mammary gland-specific expression of an oncogenic form of ErbB-2 resulted in rapid induction of multifocal mammary tumors

It is expected that the methods of the invention can also be used to treat malignancies associated with proteins that are similar to ErbB-2, e.g., other ErbB family members, and more generally with malignancies associated with growth factor receptors. ErbB1 has been causally implicated in human malignancy, e.g., aggressive carcinomas of the breast, bladder, lung, and stomach. ErbB gene amplification or overexpression, or a combination of both, has also been demonstrated in squamous cell carcinomas and glioblastomas (Libermann, T. A., Nusbaum, H. R., Razon, N., Kris, R., Lax, I., Soreq, H., Whittle, N., Waterfield, M. D., Ulhrich, A. & Schlessinger, J., 1985, Nature 313:144-147). Accordingly, the agents of the invention are believed to be useful for treating these malignancies. ErbB3 has been found to be overexpressed in breast (Lemoine et al., Br. J. Cancer 66:1116-21 (1992)), gastrointestinal (Poller et al., J. Pathol. 168:275-80 (1992); Rajkumer et al., J. Pathol. 170:271-78 (1993); Sanidas et al., Int. J. Cancer 54:935-40 (1993)), and pancreatic cancers (Lemoine et al., J. Pathol. 168:269-73 (1992), and Friess et al., Clinical Cancer Research 1:1413-20 (1995)). Plowman et al. found that Increased erbB4 expression have been found to closely correlate with certain carcinomas of epithelial origin, including breast adenocarcinomas (Plowman et al., PNAS 90:1746-50 (1993) and Plowman et al., Nature 366:473-75 (1993)).

Other types of proliferative disorders that can be treated according to the invention include non malignant cell proliferative disorders, such as those associated with an abnormal production of, or response to a growth factor, e.g., platelet derived growth factor (PDGF), fibroblast derived growth factor (FGF), epidermal derived growth factor (EGF) and vascular endothelial growth factor (VEGF). Exemplary diseases include restinosis, glomerulonephritis, neurofibromatosis, glaucoma, psoriasis, rheumatoid arthritis, inflammatory bowel disease, and chemotherapy-induced alopecia and mucositis.

In another embodiment, the agents of the invention are used for treating inflammatory diseases, e.g., rheumatoid arthritis (R.A.). Synovial tissues of RA patients express high levels of FGF and PDGF compared with synovial tissues of osteoarthritis patients, a non invasive joint disease (Sano et al., J. Cell. Biol. 110:1417-1426, 1990). These data are consistent with the theory that PDGF and FGF play a role in generating an invasive tumor-like behavior in arthritic joints of RA synovial connective tissues (Sano et al., J. Clin. Invest. 91:553-565 1993).

It is further expected that the agents of the invention are useful for treating smooth muscle cell hyper-proliferation, at least in part since PDGF is considered to be a principal growth-regulatory molecule responsible for smooth muscle cell proliferation. One smooth muscle disorder is atherosclerosis, which is a disease characterized by focal thickening of the inner portion of the artery wall, predisposing an individual to myocardial infarction (heart attack), cerebral infarction (stroke), hypertension (high blood pressure) and gangrene of the extremities. In addition to consisting primarily of proliferated smooth muscle cells, lesions of atherosclerosis are surrounded by large amounts of lipid-laden macrophages, varying numbers of lymphocytes and large amounts of connective tissue. PDGF has been found in numerous cells in such lesions, and it is believed that PDGF plays a critical role in the atherosclerosis disease process. Other smooth muscle diseases include diabetic vascular pathologies.

Both FGF and VEGF are potent angiogenic factors which induce formation of new capillary blood vessels. Accordingly, the agents of the invention may be useful in inhibiting vascularization, e.g., in tumors.

In addition, the instant agents may also be useful in the treatment of certain viral infections, in particular in the treatment of hepatitis C or delta and related viruses (J. S. Glenn et al. Science, 256:1331-1333 (1992)). Numerous viruses also induce non cancerous cell proliferation. Examples include papilloma viruses (HPV), which create skin lesions. Such viral infections may also be treatable with the compositions of the invention.

The agents of the invention can also be used for treatment of hyperproliferative cutaneous diseases, e.g., keratosis and psoriasis.

### 9. Administration of Agents to Cells and Subjects

The therapeutic methods of the invention generally comprise administering to a subject in need thereof, a pharmaceutically effective amount of an agent. The agent can be a macromolecule, e.g., a nucleic acid (e.g., an antisense or dsRNA nucleic acid), peptide, or small organic molecule. The agents of this invention may be administered to mammals, preferably humans, either alone or, preferably, in combination with pharmaceutically acceptable carriers, excipients or diluents, in a pharmaceutical composition, according to standard pharmaceutical practice. The agents can be administered orally or parenterally, including the intravenous, intramuscular, intraperitoneal, subcutaneous, rectal and topical routes of administration.

Toxicity and therapeutic efficacy of the agents can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents which exhibit large therapeutic indices are preferred. While agents that exhibit toxic side effects may be used, care should be taken to design a delivery system that targets such reagents to the'site of affected tissue in: order to minimize potential damage to normal cells and, thereby, reduce side effects.

Data obtained from cell culture assays and animal studies can be used in formulating a range of dosage for use in humans. The dosage of such reagents lies preferably within a range of circulating concentrations that include the ED₅₀ with little or no toxicity. The dosage may vary within this range depending upon the dosage form employed and the route of administration utilized. For any reagent used in the method of the invention, the therapeutically effective dose can be estimated initially from cell culture assays. A dose may be formulated in animal models to achieve a circulating plasma concentration range that includes the IC₅₀ (i.e., the concentration of the test agent which achieves a half-maximal inhibition of symptoms) as determined in cell culture. Levels of agents in plasma may be measured, for example, by high performance liquid chromatography.

Pharmaceutical compositions containing an agent of the invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of, aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the agent of the invention in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solutions. Among the acceptable vehicles and solvents that may be employed are water, Ringer's solution and isotonic sodium chloride solution.

Sterile injectable preparation may also be a sterile injectable oil-in-water microemulsion where the agent of the invention is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant agent. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Agents of the invention may also: be administered in the form of a suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the agent of the invention can be employed. For purposes of this application, topical application shall include mouth washes and gargles.

The agents for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will preferably be continuous rather than intermittent throughout the dosage regimen.

The agents of the invention may also be co-administered with other well known therapeutic agents that are selected for their particular usefulness against the condition that is being treated. The agents may be administered simultaneously or sequentially. For example, the instant agents may be useful in combination with known anti-proliferative agents, e.g., anti-neoplastic drugs. Methods for the safe and effective administration of anti-proliferative agents are known to those skilled in the art. In addition, their administration is described in the standard literature. For example, the administration of many of the anti-proliferative agents is described in the “Physicians Desk Reference” (PDR), e.g., 1996 edition (Medical Economics Company, Montvale, N.J. 07645-1742, USA).

When a composition according to this invention is administered into a human subject, the daily dosage will normally be determined by the prescribing physician with the dosage generally varying according to the age, weight, and response of the individual patient, as well as the severity of the patient's symptoms.

### 10. Diagnostic Methods

In certain embodiments, the invention also provides diagnostic methods and compositions for detecting an HCAP-BP, e.g., ErbB-2. For example, the invention provides HCAP or portions thereof, which are optionally labeled, and which are used to detect HCAP-BP, e.g., ErbB-2. The invention also provides diagnostic methods and compositions for detecting HCAP. For example, the invention provides an HCAP-BP, e.g., ErbB-2, or portions thereof, which are optionally labeled, and which are used to detect HCAP.

In one embodiment, the detection methods comprise contacting a sample, such as a tissue sample with a detection agent (e.g., a portion of HCAP for detecting an HCAP-BP or a portion of an HCAP-BP for detecting HCAP) in conditions under which the HCAP and HCAP-BP or portions thereof interact. The detection agent may be labeled with a label, e.g., a fluorescent or radioactive label, as known in the art. The sample can be a biopsy from a patient, e.g., a breast cancer patient. The sample can be a cell or tissue sample, or a cell or protein lysate.

### 11. Kits

In one embodiment, an agent of the invention, and materials and/or reagents required for administering the agents of the invention may be assembled together in a kit. When the components of the kit are provided in one or more liquid solutions, the liquid solution preferably is an aqueous solution, with a sterile aqueous solution being particularly preferred.

The kit may further comprise one or more other agent of the invention or other drug, e.g., an anti-proliferative agent. These normally will be a separate formulation, but may be formulated into a single pharmaceutically acceptable composition. The container means may itself be geared for administration, such as an inhalant, syringe, pipette, eye dropper, or other such like apparatus.

Kits can also comprise an HCAP or HCAP-BP or portion thereof, which may be labeled, and which can be used for diagnostic purposes.

The compositions of these kits also may be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the agent.

The kits of the present invention may include a means for containing the vials in close confinement for commercial sale such as, e.g., injection or blow-molded plastic containers into which the desired vials are retained. Irrespective of the number or type of containers, the kits of the invention also may comprise, or be packaged with a separate instrument for assisting with the injection/administration or placement of the agent within the body of an animal. Such an instrument may be an inhalant, syringe, pipette, forceps, measured spoon, eye dropper or any such medically approved delivery vehicle. Other instrumentation includes devices that permit the reading or monitoring of reactions or amounts of agents.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Acadernic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

The present invention is further illustrated by the following examples which should not be construed as limiting in any way. The contents of all cited references (including literature references, issued patents, published patent applications as cited throughout this application) are hereby expressly incorporated by reference.

### Examples

### Example 1

HCAP is Associated with ErbB-2

Results:

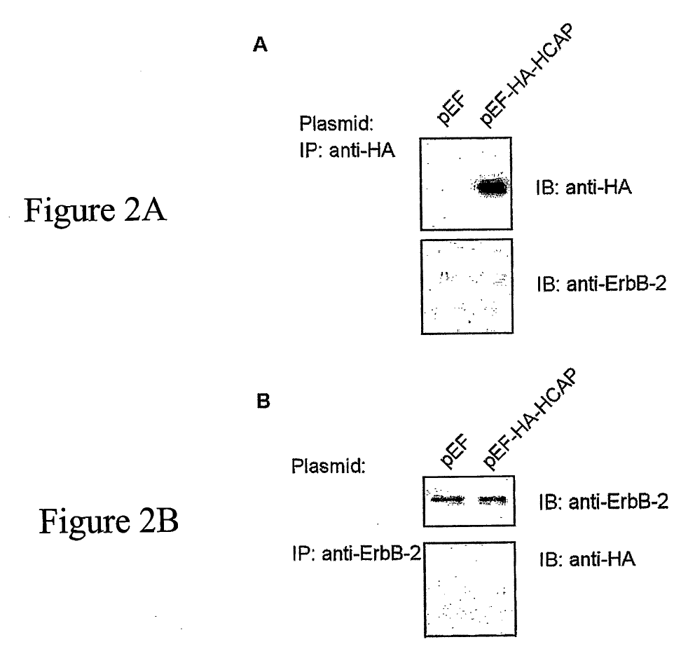

SKBR-3 cells (a breast tumor cell line) were transfected with control vector (pEF) or with a vector encoding full-length HCAP (PEF-HA-HCAP), with an HA-tag at the N-terminus. Cell extracts were immuno-precipitated with an antibody against the HA tag (FIG. 2A) or against the N-terminal region of endogenous ErbB-2 (FIG. 2B). Immuno-precipitated material was separated on SDS-PAGE and immuno-blotted with either anti-HA or Anti-ErbB-2 antibodies. As shown in FIGS. 2A and 2B, HCAP associates with ErbB-2.

Methods:

Transfection: The day before transfection SKBR-3 cells were plated at 3×10⁶ cells per 10 cm dish in growth medium (DMEM high glucose containing 10% FBS). Two hours prior to transfection the medium was replaced with 10 ml fresh growth medium. Transfection was performed with the Lipofectamin 2000 (Gibco-BRL) reagent with 3 μg plasmid DNA per plate. The cells were harvested 24 hours post-transfection.

Immunoprecipitation: Cells were washed twice with ice cold PBS and scraped in 1.5 ml PBS. Cells were solubilized in 0.5 ml sample buffer containing protease inhibitors (50 mM HEPES-NaOH, pH 7.5, 150 mM NaCl, 87% glycerol, 1 mM EDTA, 1 mM EGTA, 1.5 mM MgCl₂, 2 mM DTT and 1% Triton X-100) and incubated on ice for 30 minutes. The insoluble material was spun for 10 min at 4° C., 20,000×g. The supernatant was removed to a fresh tube and antibody conjugated to agarose beads was added. For immunoprecipitation of ErbB-2, Ab-4 was used (NeoMarkers, MS-301-PABX, Lab Vision Corp., California). Immunoprecipitation was performed for 2 hours at 4° C. At the end of incubations, beads were washed twice with high salt buffer (50 mM Tris-HCl, pH 7.5, 500 mM NaCl, 0.1% SDS, 0.1% Triton X-100, 5 mM EDTA and 5 mM EGTA), twice with medium salt buffer (50 mM Tris-HCl, pH 7.5, 150 mM NaCl, 0.1% SDS, 0.1% Triton X-100 and 5 mM EDTA) and twice with low salt buffer (50 mM Tris-HCl, pH 7.5, 0.1% Triton X-100 and 5 mM EDTA). After the last wash beads were transferred to a fresh tube and SDS-sample buffer was added. Proteins were separated on 10% SDS-PAGE and transferred to nitrocellulose membrane. Gels were western blotted with anti-ErbB-2 antibody (Santa Cruz Biotechnology, Inc., California, Cat SC-284) or with anti-HA-peroxidase.

### Example 2

Radicicol Disrupts the HCAP Interaction with ErbB-2

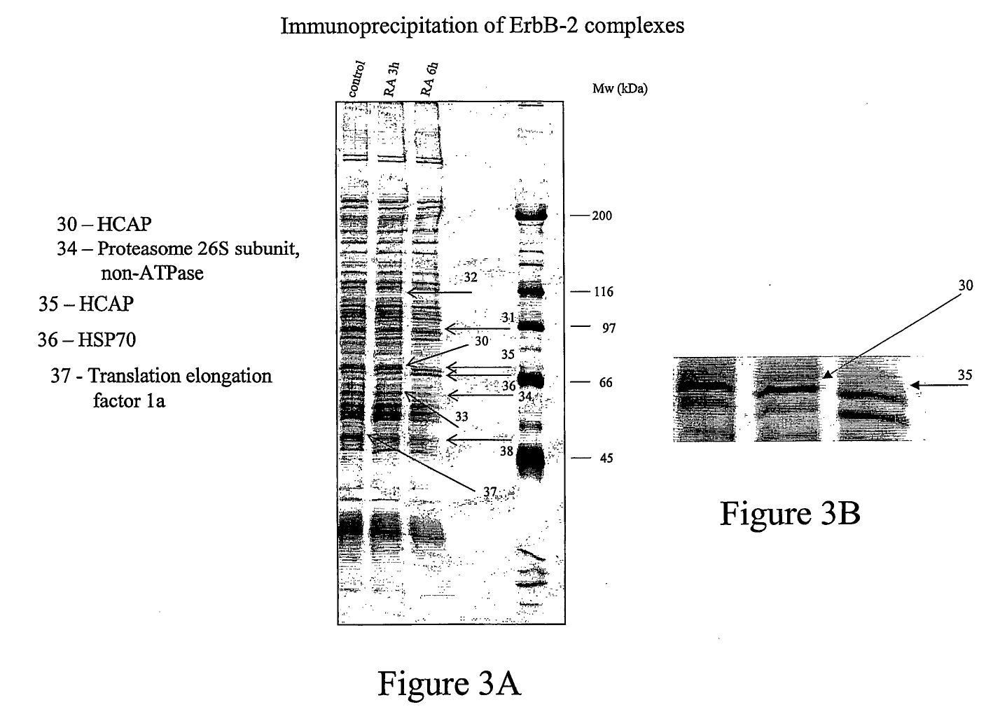

T47D cells, a breast cancer cell line that is mutant for the p53 tumor suppressor gene and ErbB-2 positive, were treated with radicicol (RA) for 0, 3 or 6 hours, and then proteins were immunoprecipitated using an anti-ErbB-2 antibody and resolved by gel electrophoresis. FIG. 3A shows the proteins resulting from the immunoprecipitation, and certain protein bands are labeled by number. Band nos. 30 and 35 were identified by mass spectroscopic analysis to be hepatocarcinoma associated protein (HCAP). The amount of HCAP immunoprecipitated with anti-ErbB-2 antibody decreased in the presence of RA.

### Example 3

Inhibition of HCAP Causes a Change in ErbB-2 Localization

Results:

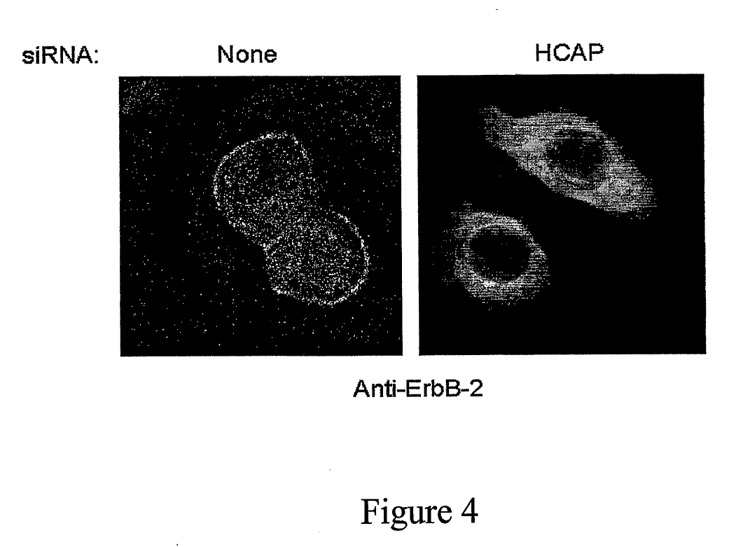

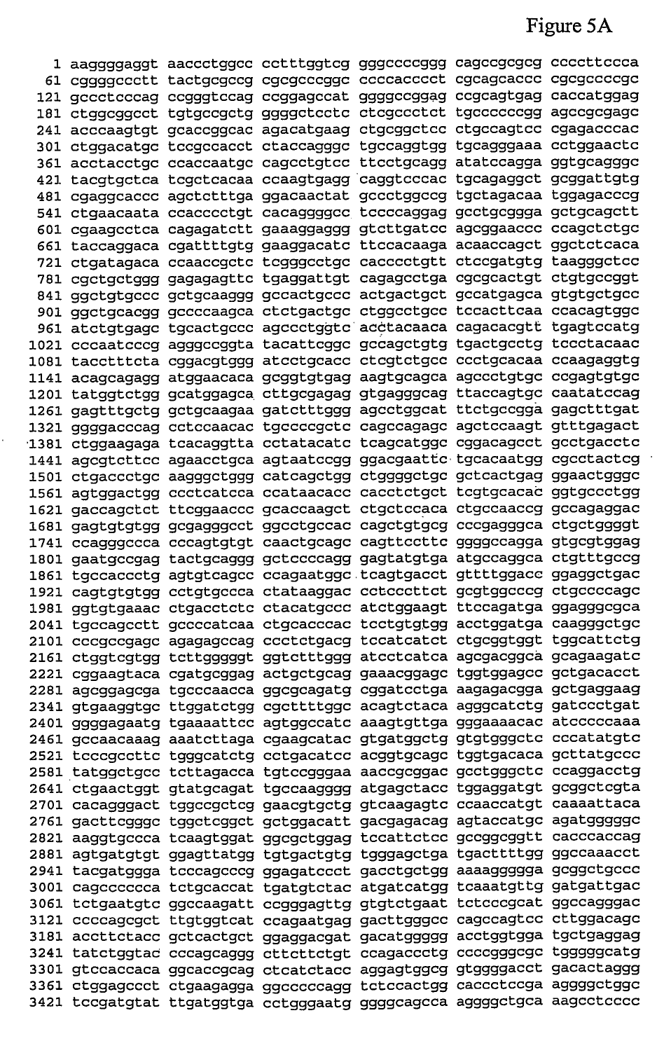

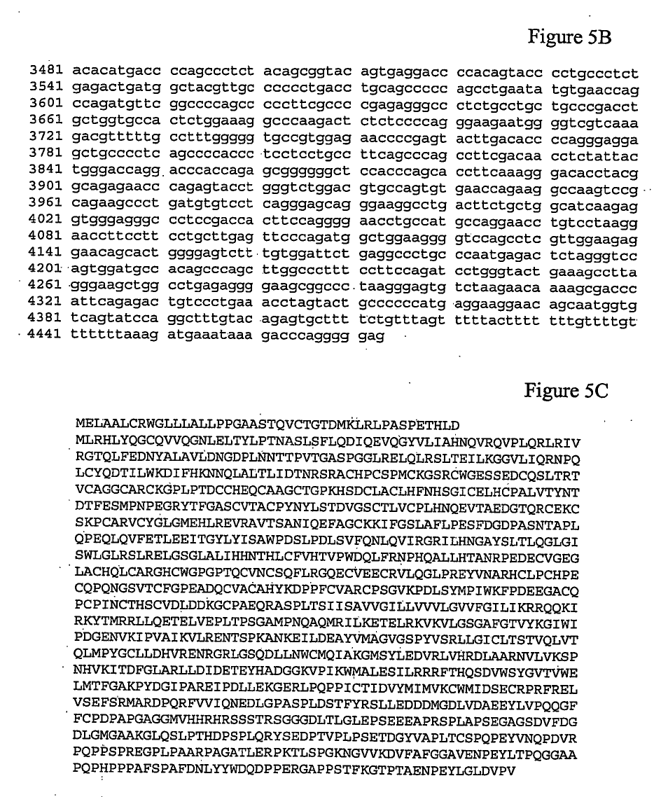

An siRNA targeted to HCAP was introduced into SKBR-3 cells grown on glass cover slips. 72 hours later, cells were fixed and stained with anti-ErbB-2 antibody. As shown in FIG. 4, control cells show significant localization of ErbB-2 at the cell membrane, as can be seen by the staining of the outline of the cell. In cells treated with HCAP siRNA, ErbB-2 is no longer seen at the cell membrane, and is instead distributed in the cytoplasm.

Methods:

Transfection: The day before transfection SKBR-3 cells were plated at 3×10⁶ cells per 10 cm dish in growth medium (DMEM high glucose containing 10% FBS). Two hours prior to transfection the medium was replaced with 10 ml fresh growth medium. Transfection was performed with the Lipofectamin 2000 (Gibco-BRL) reagent with 50 nM siRNA per plate. The cells were harvested 24 hours post-transfection. siRNA probes targeted to (containing a strand complementary to) the following HCAP sequences were used:

| | |
|---|---|
| Name | Sequence targeted |

| | | |
|---|---|---|
| HCAP-351 | AATGCCTGCCACTGAGACCAA | (SEQ ID No. 5) |
| HCAP-1303 | AATCCCCCTGAATATGAGTTC | (SEQ ID No. 6) |
| HCAP-1409 | AATGGGCAGCTCAGTACCGAG | (SEQ ID No. 7) |

Immunofluorescence: SKBR cells were plated on 35 mm dishes. Cells were washed three times with PBS (10 minutes each wash). Cells were fixed in fixation solution (1% BSA, 1% natural goat serum, 0.5% gelatin, 2% formaldehyde in PBS pH 7.3) for 30 minutes at 4° C. Subsequently, cells were washed three times with PBS and permeabilized with the following solution: 1% BSA, 1% natural goat serum, 0.5% gelatin, 0.5% Triton X-100 in PBS pH 7.3. Cells were washed three times with wash buffer (0.1% BSA, 0.1% natural goat serum, 0.05% gelatin and 0.25% Tween 20 in PBS, pH 7.3). Anti-ErbB-2 N-terminal antibody was added at a dilution of 1:500 in wash buffer. Incubation was performed overnight at 40° C. The cells were washed three times with wash buffer and secondary Cy2-conjugated antibody was added at a 1:500 dilution in wash buffer. Cells were washed three times with wash buffer and analyzed by confocal microscopy.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for treating an HCAP associated disease in a subject, comprising administering to the subject a pharmaceutically effective amount of an agent which inhibits the interaction between HCAP and an HCAP binding partner (HCAP-BP).

2. The method of claim 1, wherein the HCAP-BP is a cell membrane receptor.

3. The method of claim 2, wherein the membrane receptor is a growth factor receptor.

4. The method of claim 3, wherein the growth factor receptor is a member of the Erb/HER family of receptors.

5. The method of claim 1, wherein the HCAP-BP is ErbB-2.

6. The method of claim 1, wherein the agent is a small organic molecule.

7. The method of claim 1, wherein the agent mimics an HCAP recognition site on HCAP-BP.

8. The method of claim 7, wherein the agent mimics an HCAP recognition site on ErbB-2.

9. The method of claim 1, wherein the agent mimics an HCAP-BP recognition site on HCAP.

10. The method of claim 9, wherein the agent mimics an ErbB-2 recognition site on HCAP.

11. A method for treating an ErbB-2 associated disease in a subject, comprising administering to the subject a pharmaceutically effective amount of an agent which decreases the level and/or activity of HCAP in cells of a subject.

12. The method of claim 11, wherein the agent is a nucleic acid which decreases the level of HCAP RNA or protein.

13. The method of claim 12, wherein the agent is an HCAP specific ribozyme.

14. The method of claim 12, wherein the agent is an HCAP specific antisense molecule.

15. The method of claim 12, wherein the agent is a dsRNA.

16. The method of claim 12, wherein the agent forms a triplex with the HCAP gene.

17. The method of claim 11, wherein the agent is a small molecule that prevents the recruitment of HCAP to the plasma membrane.

18. The method of claim 11, wherein the agent is a small molecule that induces the degradation of HCAP protein.

19. The method of claim 1 or 11, wherein the disease is a cell proliferative disease.

20. The method of claim 19, wherein the cell proliferative disease is a cell hyperproliferative disease.

21. The method of claim 20, wherein the cell hyperproliferative disease is cancer.

22. The method of claim 21, wherein cancer is an ErbB-2 associated cancer.

23. The method of claim 22, wherein the ErbB-2 associated cancer is breast cancer.

24. The method of claim 20, wherein the cell hyperproliferative disease is a benign proliferative disease.

25. A method for inhibiting proliferation of a cell, comprising contacting the cell with an agent which inhibits the interaction between HCAP and an HCAP-BP, such that the proliferation of the cell is inhibited.

26. The method of claim 25, wherein the HCAP-BP is a growth factor receptor.

27. The method of claim 26, wherein the growth factor receptor is ErbB-2.

28. A composition comprising an isolated HCAP and an isolated HCAP-BP.

29. The composition of claim 28, wherein HCAP-BP is a growth factor receptor.

30. The composition of claim 29, wherein the growth factor receptor is ErbB-2.

31. An isolated protein complex comprising HCAP and an HCAP-BP.

32. The complex of claim 31, wherein HCAP-BP is a growth factor receptor.

33. The composition of claim 32, wherein the growth factor receptor is ErbB-2.

34. A method for identifying an agent which modulates the interaction between an HCAP polypeptide and an HCAP-BP, comprising contacting an isolated HCAP polypeptide with an isolated HCAP-BP in the presence of a test agent under conditions in which, but for the presence of the test agent, the HCAP polypeptide interacts with HCAP-BP, such that a different level of HCAP polypeptide and HCAP-BP complex in the presence of the test agent relative to the absence of the test agent indicates that the test agent modulates the interaction between the HCAP polypeptide and the HCAP-BP.

35. The method of claim 34, wherein the HCAP-BP comprises an amino acid sequence that is at least 99% identical to a fragment of SEQ ID No. 4 that is at least 25 amino acids in length and binds to the HCAP polypeptide.

36. The method of claim 34, wherein the HCAP polypeptide comprises an amino acid sequence that is at least 99% identical to a fragment of SEQ ID No. 2 that is at least 25 amino acids in length and binds to the HCAP-BP.

37. An isolated or recombinant polypeptide complex comprising an HCAP polypeptide and an Erb/Her receptor family polypeptide, wherein the HCAP polypeptide comprises an amino acid sequence that is at least 90% identical to the amino acid sequence of SEQ D No. 2, and wherein the Erb/Her receptor family polypeptide comprises an amino acid sequence that is at least 90% identical to the amino acid sequence of SEQ ID No. 4.

38. The polypeptide complex of claim 37, wherein the Erb/Her receptor family polypeptide comprises an amino acid sequence that is at least 95% identical to the amino acid sequence of SEQ ID No. 4.

39. The polypeptide complex of claim 37, wherein the HCAP polypeptide further comprises an additional moiety selected from the group consisting of: an epitope tag, a purification moiety and a detection moiety.

40. The polypeptide complex of claim 37, wherein the Erb/Her receptor family polypeptide further comprises an additional moiety selected from the group consisting of: an epitope tag, a purification moiety and a detection,moiety.

41. An isolated or recombinant polypeptide complex comprising an HCAP polypeptide and an Erb/Her receptor family polypeptide, wherein the HCAP polypeptide comprises an amino acid sequence that is at least 99% identical to a fragment of SEQ ID No. 2 that is at least 25 amino acids in length and binds to the Erb/Her receptor family polypeptide, and wherein the Erb/Her receptor family polypeptide comprises an amino acid sequence that is at least 90% identical to a cytoplasmic domain of SEQ ID No. 4.

42. An isolated or recombinant polypeptide complex comprising an HCAP polypeptide and an Erb/Her receptor family polypeptide, wherein the HCAP polypeptide comprises an amino acid sequence that is at least 90% identical to SEQ ID No. 2, and wherein the Erb/Her receptor family polypeptide comprises an amino acid sequence that is at least 99% identical to a fragment of SEQ ID No. 4 that is at least 25 amino acids in length and binds to the HCAP polypeptide.

43. A double stranded RNA comprising a sequence that is at least 90% identical to a sequence of at least 15 nucleotides of SEQ ID No. 1.

44. A composition for administration to a human subject comprising the double stranded RNA of claim 43 and a pharmaceutically acceptable excipient.

45. A method of treating a proliferative disorder, comprising administering a composition comprising a double stranded RNA of claim 43.

46. The method of claim 45, wherein the proliferative disorder is breast cancer.

47. The method of claim 45, wherein the proliferative disorder is ErbB-2-related breast cancer.

48. The method of claim 45, wherein the proliferative disorder is taxol-resistant breast cancer.
